(19) United States
(12) Patent Application Publication
(10) Pub. No.: US 2022/0130496 A1
(43) Pub. Date: Apr. 28, 2022

(54) METHOD OF TRAINING PREDICTION MODEL FOR DETERMINING MOLECULAR BINDING FORCE

(71) Applicant: Baidu.com Times Technology (Beijing) Co., Ltd., Beijing (CN)

(72) Inventors: Shuangli LI, Beijing (CN); Jingbo ZHOU, Beijing (CN); Tong XU, Hefei (CN); Liang HUANG, Beijing (CN); Fan WANG, Beijing (CN); Haoyi XIONG, Beijing (CN); Weili HUANG, Eugene, OR (US); Hui XIONG, Kowloon (HK); Dejing DOU, Beijing (CN)

(21) Appl. No.: 17/570,416

(22) Filed: Jan. 7, 2022

(30) Foreign Application Priority Data
May 18, 2021 (CN) 202110542307.4

(51) Int. Cl. G16C 20/70; G16C 20/30

(52) U.S. Cl. CPC G16C 20/70; G16C 20/30

(57) ABSTRACT

A method of training a prediction model for determining molecular binding force is provided, which relates to the field of artificial intelligence, in particular to a graph neural network in the field of deep learning. The method includes: constructing a virtual complex molecule based on a three-dimensional structure information of a first molecule and a second molecule; determining a predicted binding force and a predicted interaction matrix between the first molecule and the second molecule based on the virtual complex molecule by using the prediction model, the predicted interaction matrix indicating an element-type-based and distance-based interaction between an atom in the first molecule and an atom in the second molecule; and training the prediction model by minimizing a target loss function based on a difference between the predicted binding force and a real binding force and a difference between the predicted interaction matrix and a real interaction matrix.

### CROSS-REFERENCE TO RELATED APPLICATION

This application claims the benefit of Chinese Patent Application No. 202110542307.4 filed on May 18, 2021, the whole disclosure of which is incorporated herein by reference.

### TECHNICAL FIELD

The present disclosure relates to the field of artificial intelligence technology, and in particular to a graph neural network in the field of deep learning. More specifically, the present disclosure relates to a method of training a prediction model for determining molecular binding force, an electronic device, a computer-readable storage medium, and a computer program product.

### BACKGROUND

In the fields of computational biology and computational chemistry, an effective prediction of molecular binding force is crucial to the understanding of biochemical properties of complexes (also known as complex molecules). For example, a protein-ligand binding force may reflect a degree of a binding reaction between the two, that is, an effectiveness of the ligand for protein. Therefore, the effective prediction of molecular binding force may help screen new drugs, accelerate drug development and reduce research and development costs.

### SUMMARY

The present disclosure provides a method of training a prediction model for determining molecular binding force, a device, and a storage medium.

According to a first aspect of the present disclosure, a method of training a prediction model for determining molecular binding force is provided, and the method includes: constructing a virtual complex molecule based on a three-dimensional structure information of a first molecule and a second molecule, the virtual complex molecule includes a virtual representation of the first molecule and a virtual representation of at least a part of the second molecule; determining a predicted binding force between the first molecule and the second molecule and a predicted interaction matrix between the first molecule and the second molecule based on the virtual complex molecule by using the prediction model, the predicted interaction matrix indicates an element-type-based and distance-based interaction between an atom in the first molecule and an atom in the second molecule; and training the prediction model by minimizing a target loss function based on a difference between the predicted binding force and a real binding force and a difference between the predicted interaction matrix and a real interaction matrix.

According to another aspect of the present disclosure, an electronic device is provided, and the electronic device includes: at least one processor; and a memory communicatively connected to the at least one processor, the memory stores instructions executable by the at least one processor, and the instructions, when executed by the at least one processor, cause the at least one processor to implement the method according to the first aspect of the present disclosure.

According to another aspect of the present disclosure, a non-transitory computer-readable storage medium having computer instructions therein is provided, the computer instructions are configured to cause a computer to implement the method according to the first aspect of the present disclosure.

It should be understood that the content described in this part is not intended to identify critical or important features of the embodiments of the present disclosure, and it is not intended to limit the scope of the present disclosure. Other features of the present disclosure may become easily understood according to the following description.

### BRIEF DESCRIPTION OF THE DRAWINGS

The accompanying drawings are used to better understand the solution and do not constitute a limitation to the present disclosure, in which:

FIG. 1 shows an architecture diagram of a system of training a prediction model according to an embodiment of the present disclosure;

FIG. 2 shows a schematic diagram of constructing a virtual complex molecule according to an embodiment of the present disclosure;

FIG. 3 shows a schematic diagram of a combination process for edges according to an embodiment of the present disclosure;

FIG. 4 shows a schematic diagram of a combination process for atoms according to an embodiment of the present disclosure;

FIG. 5 shows a schematic diagram of a process of determining a predicted binding force and a predicted interaction matrix according to an embodiment of the present disclosure;

FIG. 6 shows a flowchart of a method of training a prediction model according to an embodiment of the present disclosure;

FIG. 7 shows a schematic block diagram of an apparatus of training a prediction model according to an embodiment of the present disclosure; and

FIG. 8 shows a block diagram of an electronic device used to implement a method of training a prediction model according to an embodiment of the present disclosure.

### DETAILED DESCRIPTION OF EMBODIMENTS

The exemplary embodiments of the present disclosure are described below with reference to the accompanying drawings, which include various details of the embodiments of the present disclosure to facilitate understanding, and which should be considered as merely illustrative. Therefore, those of ordinary skilled in the art should realize that various changes and modifications may be made to the embodiments described herein without departing from the scope and spirit of the present disclosure. In addition, for clarity and conciseness, descriptions of well-known functions and structures are omitted in the accompanying description.

As described above, in the fields of computational biology and computational chemistry, the effective prediction of molecular binding force is crucial to the understanding of the biochemical properties of complex molecules. A molecule is essentially a network structure graph composed of multiple types of atoms that interact with each other. In addition to a topological structure information, the network structure graph of the molecule also contain a key spatial structure information, for example, an angle and a distance between atoms that compose the molecule. Existing methods of determining a molecular binding force (for example, affinity) require more time and computing resources, such as methods determined by experiments or methods based on physical simulations. Therefore, there is a need for a method that may better characterize a three-dimensional structure information of a molecule and accurately predict a molecular binding force.

According to an embodiment of the present disclosure, a solution of training a prediction model for determining molecular binding force is proposed. In this solution, a virtual complex molecule is constructed based on a three-dimensional structure information of a first molecule and a second molecule. The solution further includes determining a predicted binding force between the first molecule and the second molecule and a predicted interaction matrix between the first molecule and the second molecule based on the virtual complex molecule by using the prediction model. The predicted interaction matrix indicates an element-type-based and distance-based interaction between an atom in the first molecule and an atom in the second molecule. The solution further includes training the prediction model by minimizing a target loss function based on a difference between the predicted binding force and a real binding force and a difference between the predicted interaction matrix and a real interaction matrix. In this way, the prediction model may be used to learn a long-distance interaction information between molecules, so as to better determine the molecular binding force.

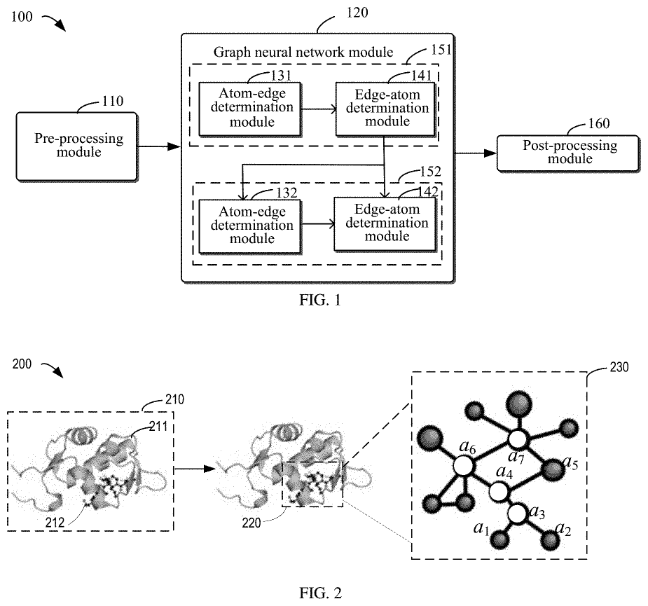

FIG. 1 shows an architecture diagram of a system 100 of training a prediction model according to an embodiment of the present disclosure. As shown in the figure, the system 100 may include a pre-processing module 110, a graph neural network module 120, and a post-processing module 160. The graph neural network module 120 may include atom-edge determination modules 131, 132 (hereinafter collectively referred to as 130) and edge-atom determination modules 141, 142 (hereinafter collectively referred to as 140). The graph neural network module may include a plurality of layers L, such as a first layer 151 and a second layer 152. The atom-edge determination module 131 and the edge-atom determination module 141 may be executed in the first layer 151. The atom-edge determination module 132 and the edge-atom determination module 142 may be executed in the second layer 152. It should be understood that the system 100 shown in FIG. 1 is only exemplary, and should not constitute any limitation to the functions and scope of the implementation described in the present disclosure. For example, the graph neural network module 120 may include more than two atom-edge determination modules and edge-atom determination modules.

The pre-processing module 110 may receive a three-dimensional structure information of combined molecules, and construct a virtual complex molecule based on the three-dimensional structure information. Hereinafter, the details of constructing the virtual complex molecule will be described with reference to FIG. 2. FIG. 2 shows a schematic diagram of constructing a virtual complex molecule according to an embodiment of the present disclosure. As shown in FIG. 2, the pre-processing module 110 may receive a three-dimensional structure information 210 of combined molecules. The combined molecules may refer to at least two molecules bound by chemical bonds, such as molecule 211 and molecule 212 shown in FIG. 2. Molecule 211 and molecule 212 may be any suitable molecules. The molecule 211 may be a protein molecule with a larger volume, and the molecule 212 may be a ligand molecule with a smaller volume.

The three-dimensional structure information 210 of combined molecules may include a three-dimensional structure information of each of the combined molecules. The three-dimensional structure information 210 of combined molecules may also include a structural interaction information of the combined molecules. The three-dimensional structure information of each molecule of the combined molecules may include a type and spatial distribution of an atom composing the each molecule. Additionally, the three-dimensional structure information of each molecule may also include a type, physical and chemical properties, name, etc. of the molecule itself. The structural interaction information of the combined molecules may be an information describing a relative structure and interaction between molecules. For example, the structural interaction information of the combined molecules may include relative spatial positions of the molecule 211 and the molecule 212. The structural interaction information of the combined molecules may also include an information of a chemical bond formed by an atom in the molecule 211 and an atom in the molecule 212. The three-dimensional structure information 210 of combined molecules may be in a form of a molecular graph, or any form that may represent the three-dimensional structure information 210 of the combined molecules.

The pre-processing module 110 may construct a virtual complex molecule based on the three-dimensional structure information 210 of combined molecules. The virtual complex molecule includes a virtual representation of the first molecule and a virtual representation of at least a part of the second molecule. In the context of the present disclosure, the terms “first molecule” and “second molecule” may refer to real molecular compounds, and the term “virtual representation of a molecule” may refer to a virtual molecular compound implemented by a computer. It should be understood that the terms “molecule” and “virtual representation of a molecule” may be used interchangeably in certain contexts of the present disclosure. For example, the virtual complex molecule may include a virtual representation of the ligand molecule 212 and a virtual representation of a part of the protein molecule 211, thereby reducing an amount of calculation and saving calculation resources. It should be understood that the virtual complex molecule may also include a virtual representation of a part of the ligand molecule 212 and a virtual representation of a part of the protein molecule 211.

In some embodiments, the pre-processing module 110 may construct a virtual complex molecule including a virtual representation of the first molecule and a virtual representation of at least a part of the second molecule. The pre-processing module 110 may determine a distance between an atom in the molecule 211 and an atom in the molecule 212 based on the structural interaction information in the three-dimensional structure information 210. For example, the pre-processing module 110 may determine a distance between a target atom in the protein molecule 211 and an atom in the ligand molecule 212. If it is determined that the distance between the target atom and any atom in the ligand molecule 212 is less than a first threshold, the pre-processing module 110 may combine the target atom with all the atoms in the ligand molecule 212 to serve as the atoms of the virtual complex molecule. In this case, all atoms in the ligand molecule 212 are determined to be atoms of the virtual complex molecule. The first threshold may be a preset value, for example, 5 angstroms. In some embodiments, the distance between the target atom in the molecule 211 and a central atom in the molecule 212 may also be determined. The central atom may be a pre-designated atom. If it is determined that the distance between the target atom in the molecule 211 and the central atom in the molecule 212 is less than the threshold, the target atom and all the atoms in the ligand molecule 212 may be combined to serve as the atoms of the virtual complex molecule.

As shown in FIG. 2, the ligand molecule 212 and a part of the protein molecule 211 included in the virtual complex molecule are shown in a dashed box 220. In this way, by adding only atoms in the protein molecule 211 that are closer to the ligand molecule 212 to the virtual complex molecule, it is possible to ignore atoms in the protein molecule 211 that have small interaction due to their long distance from the ligand molecule 212, so that the prediction model may be used to better predict the interaction between molecules, such as molecular binding force (also known as molecular affinity).

The pre-processing module 110 may also construct an edge between atoms of the virtual complex molecule. Specifically, it is possible to determine an atomic pair having a distance less than a second threshold among the atoms of the virtual complex molecule, and construct an edge for the atomic pair. The second threshold may be a preset value, for example, 3 angstroms. FIG. 2 shows an exemplary constructed virtual complex molecule 230. As shown in the figure, the virtual complex molecule 230 may include atoms a3, a4, a6, and a7 from the ligand molecule 212 shown in a light color and atoms a1, a2 and a5 from the protein molecule 211 shown in a dark color. FIG. 2 also shows edges constructed in the virtual complex molecule 230. It may be seen from the figure that the pre-processing module 110 does not construct edges between atoms that are far apart (for example, a6 and a2).

With reference to FIG. 1, the pre-processing module 110 may determine an initial representation of an atom in the virtual complex molecule 230 and an initial representation of an edge in the virtual complex molecule 230 based on a three-dimensional structure information of the virtual complex molecule 230. The three-dimensional structure information of the virtual complex molecule 230 may include an information related to an atom in the virtual complex molecule 230 in the three-dimensional structure information 210 of the combined molecules. The initial representation of the atom in the virtual complex molecule 230 may be an initial vector representation generated based on information such as a property of the atom, a spatial distribution, and a property of the molecule. Various methods may be used to determine the initial representation of the atom, and the scope of the present disclosure does not limit this.

The pre-processing module 110 may also determine the initial representation of the edge in the virtual complex molecule 230 based on the three-dimensional structure information of the virtual complex molecule. The pre-processing module 110 may determine a characterization of a distance between atoms connected by the edge based on the three-dimensional structure information of the virtual complex molecule. In some embodiments, the characterization of the distance may be obtained by vectorizing the distance between atoms. For example, the distance between atoms may be discretized to obtain a one-hot encoding of the distance. The characterization of the distance may be obtained based on the one-hot encoding of the distance.

The pre-processing module 110 may also determine an angle between neighboring edges connecting to a same atom based on the three-dimensional structure information of the virtual complex molecule. In some embodiments, a polar coordinate system may be used to represent the three-dimensional structure information of the virtual complex molecule. In this case, it is easier to calculate the angle between neighboring edges. For example, a first edge connecting to a first atom may be used as a polar axis, and the first atom may be used as a pole. The pre-processing module 110 may determine an angle between a remaining edge except the first edge among the neighboring edges connecting to the first atom and the first edge. In some embodiments, the angle may be represented by (θ, φ), and the θ and φ may be within a range of 0° to 180°.

The pre-processing module 110 may input the initial representation of the atom, the characterization of the distance between the atoms, and the angle, which are determined based on the three-dimensional structure information of the virtual complex molecule, into the graph neural network module 120. The graph neural network module 120 may be a graph neural network that outputs a feature representation of the virtual complex molecule based on the above described input data.

In some embodiments, the atom-edge determination module 131 may determine an initial representation of an edge connecting atoms based on the initial representations of the atoms and the characterization of the distance between the atoms. The initial representation of the edge may be a one-dimensional vector representation. In some embodiments, the initial representations of the atoms connected by the edge and the characterization of the distance may be concatenated to determine the initial representation of the edge. Alternatively, an average value of the initial representations of the connected atoms and the characterization of the distance may be concatenated to determine the initial representation of the edge.

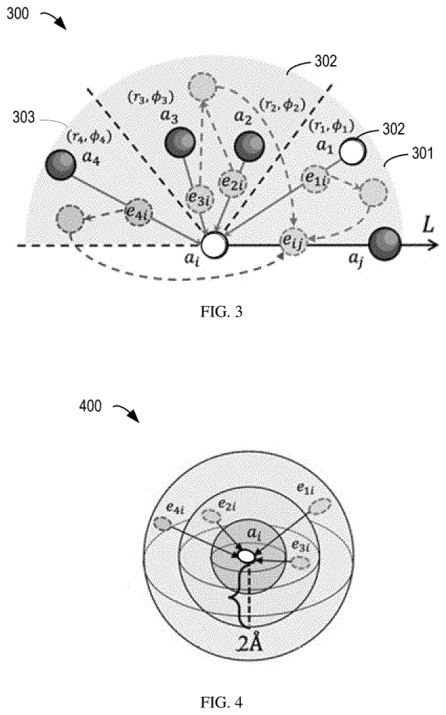

The atom-edge determination module 131 also determines a first representation of a neighboring edge based on an initial representation of the neighboring edge. Hereinafter, a combination process for edges will be described in detail with reference to FIG. 3. FIG. 3 shows a schematic diagram of a combination process 300 for edges according to an embodiment of the present disclosure. FIG. 3 shows a first atom aᵢ and neighboring edges eᵢⱼ, e₁ᵢ, e₂ᵢ, e₃ᵢ, e₄ᵢ connected to the first atom. It should be understood that aᵢ may also be used to represent a characterization of the first atom. Similarly, eᵢⱼ, e₁ᵢ, e₂ᵢ, e₃ᵢ, and e₄ᵢ may also be used to represent characterizations of the neighboring edges. The atom-edge determination module 131 determines first representations of the neighboring edges eᵢⱼ, e₁ᵢ, e₂ᵢ, e₃ᵢ, e₄ᵢ based on initial representations of the neighboring edges eᵢⱼ, e₁ᵢ, e₂ᵢ, e₃ᵢ, and e₄ᵢ of the first atom aᵢ. In some embodiments, the atom-edge determination module 131 may select a first edge eᵢⱼ from the neighboring edges, and determine a first representation of the first edge eᵢⱼ based on a combination of the initial representations of the remaining edges eₖᵢ (for example, e₁ᵢ, e₂ᵢ, e₃ᵢ, e₄ᵢ) except the first edge eᵢⱼ among the neighboring edges. The atom-edge determination module 131 may combine the initial representations of the remaining edges eₖᵢ based on angles between neighboring edges determined by the pre-processing module 110 to serve as the first representation of the first edge eᵢⱼ.

In some embodiments, the atom-edge determination module 131 may divide the remaining edges eₖᵢ into different angle domains based on the angles between neighboring edges. For example, formula (1) may be used to calculate an index Indₖᵢ of the angle domains where the remaining edges eₖᵢ are located.

$${Ind}_{ki} = {D}_{A} ( {e}_{ki} , {e}_{ij} , N ) = ⌈ N \cdot \frac{{ϕ}_{kij}}{180} ⌉ \qquad ( 1 )$$

Wherein D_{A} indicates an angle domain divider, ┌⋅┐ indicates a rounding symbol, ϕₖᵢⱼ ∈[0, 180° ] indicates an angle between an edge eₖᵢ and the edge eᵢⱼ, and N indicates a number of angle domains. As shown in FIG. 3, the remaining edges e₁ᵢ, e₃ᵢ, and e₄ᵢ are divided into angle domains 301, 302, and 303, respectively. It should be understood that the angle domain division shown in FIG. 3 is only exemplary. In some embodiments, the angle domain may be divided based on values of the angles θ and φ. It should be understood that by setting ϕₖᵢⱼ E [0, 180°], repeated combinations of a same edge may be reduced. It is also possible to set ϕₖᵢⱼ ∈[0, 360°] and other rules for the combination process of neighboring edges.

In some embodiments, the atom-edge determination module 131 may determine the attention weight(s) of the remaining edge(s) eₖᵢ in each angle domain to the first edge eᵢⱼ. For example, an attention weight of the remaining edge e₁ᵢ in the angle domain 301 to the first edge eᵢⱼ may be determined; attention weights of the remaining edges e₂ᵢ and e₃ᵢ in the angle domain 302 to the first edge eᵢⱼ may be determined; and an attention weight of the remaining edge e₄ᵢ in the angle domain 303 to the first edge eᵢⱼ may be determined. Formulas (2) and (3) may be used to calculate the attention weight(s) of the remaining edge(s) eₖᵢ in the angle domain q to the first edge eᵢⱼ.

$$\begin{matrix}{attn}_{q}^{l} ( {e}_{ij} , {e}_{ki} ) = {u}_{l , q}^{T} \cdot tan h ( {W}_{e , q}^{( l )} \cdot [ {e}_{ij}^{( l )} ∥ {e}_{ki}^{( l )} ] + {b}_{e , q}^{( l )} ) & ( 2 ) \\ {α}_{ki , q}^{( l )} = \frac{exp ( {attn}_{q}^{l} ( {e}_{ij} , {e}_{ki} ) )}{{\sum }_{{e}_{i} {ϵ𝒩}_{e}^{q} ( {e}_{ij} )} exp ( {attn}_{q}^{l} ( {e}_{ij} , {e}_{ti} ) )} & ( 3 )\end{matrix}$$

The function attn_{q}^{l} may calculate an importance coefficient of each neighbor eₖᵢ to eᵢⱼ in layer l. In the calculation of the atom-edge determination module 131, the layer l is the first layer 151. As shown in formula (2), a concatenation of eh and eᵢⱼ may be used to calculate the importance coefficient. w_{e,q}^{(l)}, b_{e,q}^{(l)}, u_{l,q}^{T} are trainable parameter matrices. α_{ki,q}^{(l)} indicates the attention weight(s) of the neighboring edge(s) eₖᵢ in the specific angle domain q. As shown in formula (3), αk_{i,q}^{(l)} may be obtained by using the softmax function to standardize the importance coefficient.

In some embodiments, based on the attention weight(s) α_{ki,q}^{(l)} of the remaining edge(s) eₖᵢ in each angle domain q to the first edge eᵢⱼ, the atom-edge determination module 131 may determine a weighted initial representation for a corresponding angle domain q by weighting and summing the initial representations of the remaining edges in each angle domain q. For example, formula (4) may be used to calculate the weighted initial representation for the angle domain q.

$${m}_{ij , q}^{( l )} = {\sum }_{{e}_{ki} ∈ {𝒩}_{e}^{q} ( {e}_{ij} )} {α}_{ki}^{{( l )}_{q}} \cdot {e}_{k i}^{( l )} , 1 \leq q \leq N \qquad ( 4 )$$

The atom-edge determination module 131 may also determine a combined characterization of the first edge eᵢⱼ, that is, the first representation of the first edge eᵢⱼ, by concatenating the weighted initial representations for each angle domain q. For example, formula (5) may be used to calculate the first representation of the first edge eᵢⱼ through a concatenation operation.

$${e}_{ij}^{( l )} = [ {m}_{ij , 1}^{( l )} ‖ {m}_{ij , 2}^{( l )} ‖ … ∥ {m}_{ij , 𝒩}^{( l 〉} ] \qquad ( 5 )$$

Similarly, the atom-edge determination module 131 may determine the first representations of all edges in the molecule. In this way, the information of the neighboring edges of each atom may be combined with the information of the edges connecting to the atom, so that the first representation of the edge may better characterize the edge and a surrounding molecular structure, thereby better characterizing the molecule.

With reference to FIG. 1, the atom-edge determination module 131 may input the determined first representation of the neighboring edge to the edge-atom determination module 141. The edge-atom determination module 141 determines a first representation of the first atom based on the first representation of the neighboring edge. Hereinafter, the combination process for atoms will be described in detail with reference to FIG. 4. FIG. 4 shows a schematic diagram of a combination process 400 for atoms according to an embodiment of the present disclosure. FIG. 4 shows the first atom aᵢ and the neighboring edges e₁ᵢ, e₂ᵢ, e₃ᵢ, and e₄ᵢ (hereinafter referred to as the neighboring edge eₖᵢ) of the first atom aᵢ. It should be understood that the neighboring edges e₁ᵢ, e₂ᵢ, e₃ᵢ, and e₄ᵢ are only exemplary.

With reference to FIG. 1, the atom-edge determination module 131 may input the determined first representation of the neighboring edge to the edge-atom determination module 141. The edge-atom determination module 141 determines the first representation of the first atom based on the first representation of the neighboring edge. Hereinafter, the combination process for atoms will be described in detail with reference to FIG. 3. FIG. 3 shows a schematic diagram of a combination process 300 for atoms according to an embodiment of the present disclosure. FIG. 3 shows the neighboring edges e₁ᵢ, e₂ᵢ, e₃ᵢ, and e₄ᵢ (hereinafter referred to as the neighboring edge eₖᵢ of the first atom aᵢ and the first atom aᵢ. It should be understood that the neighboring edges e₁ᵢ, e₂ᵢ, e₃ᵢ, and e₄ᵢ are only exemplary.

In some embodiments, the edge-atom determination module 141 may determine distances between the neighboring edges e₁ᵢ, e₂ᵢ, e₃ᵢ, and e₄ᵢ and the first atom aᵢ. The distance between the neighboring edge and the first atom aᵢ may be a distance between a second atom (atoms a₁, a₂, a₃, or a₄ shown in FIG. 2) and the first atom aᵢ connected by the neighboring edge. The edge-atom determination module 141 may determine an attention weight of a neighboring edge eₖᵢ to the first atom aᵢ based on the distance. For example, formulas (6) and (7) may be used to calculate an attention weight of the neighboring edge eₖᵢ to the first atom aᵢ.

$$\begin{matrix}{w}_{ki}^{( l )} = LeakyRelu ( {v}_{l}^{T} \cdot [ {\tilde{e}}_{ki}^{( l )} ‖ {\tilde{a}}_{i}^{( l )} ‖ {W}_{d}^{( l )} {d}_{ki} ] ) & ( 6 ) \\ {β}_{ki}^{( l )} = \frac{exp ( {w}_{ki}^{( l )} )}{{\sum }_{{e}_{ki} {ϵ𝒩}_{e} ( {a}_{i} )} exp ( {w}_{ki}^{( l )} ) )} & ( 7 )\end{matrix}$$

The function LeakyRelu may calculate an importance coefficient of each neighboring edge eₖᵢ to aᵢ in layer l. In the calculation of the edge-atom determination module 141, the layer l is the first layer 151. As shown in formula (6), the concatenation of \tilde{e}ₖᵢ^{(l)}, ãᵢ^{(l)}, and

$${W}_{d}^{( l )} {d}_{ki}$$

may be used to calculate the importance coefficient wₖᵢ^{(l)}. \tilde{e}ₖᵢ^{(l)} and ãᵢ^{(l)} are respectively a converted first representation of the neighboring edge eₖᵢ and a converted initial representation of the first atom aᵢ. By converting the first representation of the neighboring edge eₖᵢ and the initial representation of the first atom aᵢ, the first representation of the neighboring edge eₖᵢ and the initial representation of the first atom aᵢ may be converted to a same feature space, thereby achieving a subsequent concatenation operation. w_{d}^{(l)} and vₗ^{T} are trainable parameter matrices.

βₖᵢ^{(l)} indicates the attention weight of the neighboring edge eₖᵢ to the first atom aᵢ. As shown in formula (7), βₖᵢ^{(l)} may be obtained by using the softmax function to normalize the importance coefficient wₖᵢ^{(l)}. Based on the attention weight βₖᵢ^{(l)} of the neighboring edge eₖᵢ to the first atom aᵢ, the edge-atom determination module 141 may determine the first representation of the first atom aᵢ by determining a weighted average of first representations of the neighboring edges.

Additionally, the edge-atom determination module 141 may calculate the attention weight of the neighboring edge eₖᵢ to the first atom aᵢ for multiple times by using a multi-head attention algorithm. In this case, formula (8) may be used to calculate the weighted average of the first representations of the neighboring edges to determine the first representation of the first atom aᵢ.

$${a}_{i}^{( l )} = \frac{1}{C} {\sum }_{c = 1}^{C} {\sum }_{{e}_{ki} {ϵ𝒩}_{e} ( {a}_{i} )} {β}_{ki , c}^{( l )} \cdot {\tilde{e}}_{ki , c}^{( l )} \qquad ( 8 )$$

Wherein C indicates a number of attention heads.

Similarly, the edge-atom determination module 141 may determine the first representations of all atoms in the molecule. In this way, by combining the information of the neighboring edges of each atom into the first representation of the atom, the first representation of the atom may better characterize the atom and the surrounding molecular structure.

With reference to FIG. 1, by using the atom-edge determination module 131 and the edge-atom determination module 141, the angle and distance factors in the spatial distribution of atoms may be fully considered when characterizing the virtual complex molecule, so as to better characterize the virtual complex molecule. In some embodiments, the graph neural network module 120 may also use the atom-edge determination module 132 and the edge-atom determination module 142 in the second layer 152 to continue to iterate the representations of atoms and edges.

Similarly, the atom-edge determination module 132 may determine a second representation of a neighboring edge of each atom based on the first representation of the atom. For example, the atom-edge determination module 132 may concatenate first representations of atoms connected by a neighboring edge with a characterization of a distance to determine the second representation of the edge. The atom-edge determination module 132 may determine a third representation of the neighboring edge based on the second representation of the neighboring edge. For example, information of neighboring edges may be transferred to a third representation of a target edge among the neighboring edges through an angle-based combination. The edge-atom determination module 142 may determine the second representation of the first atom based on the third representation of the neighboring edge of the first atom. For example, information of the neighboring edge and a neighboring atom may be transferred to the second representation of the atom through a distance-based combination. Additionally, the graph neural network module 120 may also determine final representations of atoms and edges by using subsequent iterations in other layers. In this way, it is possible to interactively generate representations of atoms and edges, and integrate spatial structure information of atoms based on the angle-based combination and the distance-based combination, thereby better characterizing the virtual complex molecule.

With reference to FIG. 1, the post-processing module 160 may determine the predicted binding force between the molecule 211 and the molecule 212 and the predicted interaction matrix between the molecule 211 and the molecule 212 based on the virtual complex molecule 230. Hereinafter, the details of determining the predicted binding force and the predicted interaction matrix will be described in detail with reference to FIG. 5. FIG. 5 is a schematic diagram of a process 500 of determining a predicted binding force and a predicted interaction matrix according to an embodiment of the present disclosure. As shown in FIG. 5, the post-processing module 160 may include a predicted binding force determination module 503 and a predicted interaction matrix determination module 504.

In some embodiments, the predicted binding force determination module 503 may receive a representation 501 of an atom of the virtual complex molecule 230. The predicted binding force determination module 503 may determine a feature representation used to characterize the virtual complex molecule 230 based on the representation 501 of the atom. The feature representation used to characterize the virtual complex molecule 230 may be a one-dimensional vector representation. In some embodiments, the summation pooling h=Σₐ_{ᵢ}, aᵢ^{(L)} may be used to calculate the feature representation h of the virtual complex molecule. In some embodiments, the feature representation of the virtual complex molecule may be determined by calculating a maximum value of final representations aᵢ^{(L)} of all atoms. Additionally or alternatively, the feature representation of the virtual complex molecule may also be determined based on the final representations aᵢ^{(L)} of all atoms and final representations of all edges. The predicted binding force determination module 503 may determine the predicted binding force ŷ based on the feature representation of the virtual complex molecule by using a fully connected layer. The predicted binding force ŷ may be in numerical form. It should be understood that the predicted binding force determination module 503 may also determine the predicted binding force ŷ based on the representation 501 of the atom by using other layers commonly used in the field of machine learning.

In some embodiments, a first loss function may be determined based on the difference between the predicted binding force ŷ and a real binding force y measured from an experiment. For example, the first loss function may be determined based on an absolute error between the predicted binding force ŷ and the real binding force y. As shown in formula (9), the L1 loss function may be used to determine the first loss function.

$${ℒ}_{a} = {\sum }_{{𝒢}_{1} ϵ𝒟} \vert \hat{y} - y \vert \qquad ( 9 )$$

In some embodiments, the predicted interaction matrix determination module 504 may receive a representation 502 of an edge in the virtual complex molecule 230. The predicted interaction matrix determination module 504 may determine a predicted interaction matrix based on the representation 502 of the edge in the virtual complex molecule 230. The predicted interaction matrix indicates an element-type-based and distance-based interaction between an atom in the first molecule and an atom in the second molecule. The predicted interaction matrix determination module 504 may pool a representation of an edge corresponding to an atomic pair based on an element type of the atomic pair composed of an atom in the first molecule and an atom in the second molecule. For example, the predicted interaction matrix determination module 504 may pool the representation of the edge by using a pairwise interaction pooling layer (PiPool).

In some embodiments, the predicted interaction matrix determination module 504 may determine a set of element types of atoms in the molecule 211, for example {C, N, O, . . . }. Similarly, the predicted interaction matrix determination module 504 may determine a set of element types of atoms in the molecule 212, such as {C, N, O, P . . . }. A union operation may be performed on the set of element types of the atoms in the molecule 211 and the set of element types of the atoms in the molecule 212 to determine a total set T of element types. The total set T of element types may also be determined according to the periodic table of elements.

The predicted interaction matrix determination module 504 may determine, for a first group of atoms of a first element type Tₖ (for example, carbon element, k=6 in the total set T of element types determined according to the periodic table) belonging to the protein molecule 211 in the virtual complex molecule 230 and a second group of atoms of a second element type T₁ (for example, nitrogen element, l=7 in the total set T of element types determined according to the periodic table) belonging to the ligand molecule 212 in the virtual complex molecule 230, an atomic pair composed of an atom in the first group of atoms and an atom in the second group of atoms. The predicted interaction matrix determination module 504 may also determine an element value indexed by the first element type (for example, carbon element) and the second element type (for example, nitrogen element) in the predicted interaction matrix based on a weighted sum of representations of edges of the atomic pairs. Formulas (10) and (11) may be used to calculate the predicted interaction matrix.

$$\begin{matrix}{h}_{k , l} = {\sum }_{{e}_{ij} {ϵδ}_{I}} {\underbrace{δ ( τ ( {a}_{i} ) , {T}_{k} ) δ ( τ ( {a}_{j} ) , {T}_{1} )}}_{Divider} {w}_{h} {e}_{ij}^{( L )} \\ & ( 10 ) \\ {\tilde{Z}}_{kl} = \frac{exp ( {q}^{T} {h}_{k , l} )}{{\sum }_{i , j} exp ( {q}^{T} {h}_{i , j} )} & ( 11 )\end{matrix}$$

Wherein eᵢⱼ∈ε_{I} indicates an edge of the virtual complex molecule 230, τ(aᵢ) returns an element type of the atom aᵢ, τ(aⱼ) returns an element type of the atom aⱼ, and δ(⋅, ⋅) indicates the Kronecker function, if two entered values are equal, returning 1, otherwise returning 0. Divider indicates a separator used to select an atomic pair of element type (Tₖ, T₁), Wₕ and q^{T} indicate trainable parameters.

As shown in formula (10), by weighting and summing the representations of the edges of the atomic pairs of element type (Tₖ, T₁), a representation h_{k,l} of the interaction may be obtained. The representation h_{k,l} of the interaction may embody an interaction information of an atomic pair of the element type (Tₖ, T₁) in the virtual complex molecule 230. In addition, as shown in formula (11), the softmax function may be used to normalize h_{k,l} to obtain an element value \tilde{Z}ₖₗ indexed by the element type Tₖ and the element type T₁ in the predicted interaction matrix \tilde{Z}, that is, an element value of the k^{th} row and l^{th} column in the predicted interaction matrix.

In some embodiments, a second loss function may be determined based on a difference between the predicted interaction matrix \tilde{Z} and the real interaction matrix Z. The real interaction matrix Z may indicate an element-type-based and distance-based interaction between an atom in the first molecule and an atom in the second molecule. Similarly, for the first group of atoms of the first element type Tₖ in the protein molecule 211 and the second group of atoms of the second element type T₁ in the ligand molecule 212, it is possible to determine a number of atomic pair(s) composed of an atom in the first group of atoms and an atom in the second group of atoms having a distance less than a third threshold. The third threshold may be a preset value. It should be noted that, different from the determination of the predicted interaction matrix, the complete protein molecule 211 and the complete ligand molecule 212 are used here to determine the number of atomic pair(s) based on element type and distance. Formulas (12) and (13) may be used to determine the real interaction matrix.

$$\begin{matrix}n ( {T}_{k} , {T}_{l} ) = {\sum }_{{a}_{i} {ϵ𝒱}^{P}} {\sum }_{{a}_{i} {ϵ𝒱}^{L}} δ ( τ ( {a}_{i} ) , {T}_{k} ) δ ( τ ( {a}_{j} ) , {T}_{l} ) Θ ( {d}_{ρ} - {d}_{ij} ) & ( 12 ) \\ {Z}_{kl} = \frac{n ( {T}_{k} , {T}_{l} )}{{\sum }_{( {a}_{i} , {a}_{j} ) {ϵ𝒱}^{P} \times {𝒱}^{L}} Θ ( {d}_{ρ} - {d}_{ij} )} & ( 13 )\end{matrix}$$

Wherein αᵢ∈ [symbol] ^{P} indicates an atom belonging to the protein molecule 211, αⱼ∈ [symbol] ^{L} indicates an atom belonging to the ligand molecule 212, and Θ indicates a step function, if an entered value is greater than or equal to 0, returning 1; otherwise returning 0.

The Kronecker function δ in the formula (12) selects an atomic pair composed of an atom of the element type Tₖ in the protein molecule 211 and an atom of the element type T₁ in the ligand molecule 212. The step function selects an atomic pair whose atomic distance dᵢⱼ is less than or equal to a threshold d_{ρ}. Therefore, the formula (12) may be used to count a number of co-occurrences n(Tₖ, T₁) of the atomic pair based on the element type within a certain distance range. The number of co-occurrences may reflect an element-type-based and distance-based interaction between atoms from different molecules. In addition, as shown in formula (13), the number of co-occurrences n(Tₖ, T₁) may be normalized to obtain an element value indexed by the element type Tₖ and the element type T₁ in the real interaction matrix Z, that is, the element value Zₖₗ of the k^{th} row and l^{th} column in the interaction matrix.

In some embodiments, the difference between the predicted interaction matrix \tilde{Z} and the real interaction matrix Z may be used to determine the second loss function. For example, formula (14) may be used to determine the second loss function. [symbol] indicates a training data set, and F indicates a flattening operation on the matrix, that is, the matrix is converted into a vector. By reducing the second loss function, the model learning may only use an information of a part of the molecule to determine the interaction of the whole molecule.

$${ℒ}_{z} = {\sum }_{{𝒢}_{1} ϵ𝒟} ‖ F ( \tilde{Z} ) - F ( Z ) ‖ \qquad ( 14 )$$

In some embodiments, a target loss function may be determined based on a weighted sum of the first loss function and the second loss function. For example, formula (15) may be used to determine the target loss function.

[symbol]

= [symbol] ₐ+λ [symbol] _{z} (15)

The hyperparameter λ indicates a weight coefficient used to weigh the first loss function and the second loss function. In this way, by minimizing the target loss function based on the difference between the predicted interaction matrix and the real interaction matrix and the difference between the predicted binding force and the real binding force, the model may be caused to determine the binding force while determining an effect of a long-distance interaction between molecules on the binding force.

In some embodiments, the binding force between the first molecule and the second molecule may be determined based on the three-dimensional structure information of the first molecule and the second molecule by using the trained prediction model. For example, the affinity between the protein molecule 211 and the ligand molecule 212 is determined.

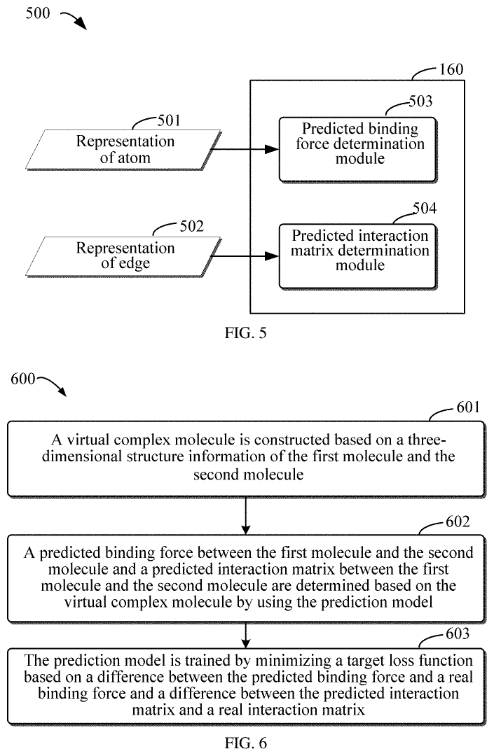

FIG. 6 shows a flowchart of a method 600 of training a prediction model according to an implementation of the present disclosure. The method 600 may be implemented on the system 100. In step 601, a virtual complex molecule is constructed based on a three-dimensional structure information of a first molecule and a second molecule. The virtual complex molecule includes a virtual representation of the first molecule and a virtual representation of at least a part of the second molecule.

In some embodiments, constructing the virtual complex molecule includes: determining a distance between a target atom in the second molecule and an atom in the first molecule based on the three-dimensional structure information; combining the target atom with the atom in the first molecule and determining the target atom and the atom in the first molecule as atoms of the virtual complex molecule, in response to determining that the distance between the target atom in the second molecule and the atom in the first molecule is less than a first threshold.

In some embodiments, constructing the virtual complex molecule includes: constructing an edge between atoms having a distance less than a second threshold in the virtual complex molecule; and determining a representation of an atom in the virtual complex molecule and a representation of the edge based on a three-dimensional structure information of the virtual complex molecule.

In step 602, a predicted binding force between the first molecule and the second molecule and a predicted interaction matrix between the first molecule and the second molecule are determined based on the virtual complex molecule by using the prediction model. The predicted interaction matrix indicates an element-type-based and distance-based interaction between an atom in the first molecule and an atom in the second molecule.

In some embodiments, determining the predicted binding force between the first molecule and the second molecule includes: determining a feature representation for characterizing the virtual complex molecule based on the representation of the atom in the virtual complex molecule; and determining the predicted binding force based on the feature representation using a fully connected layer in the prediction model.

In some embodiments, determining the predicted interaction matrix includes: determining, for a first group of atoms of a first element type in the first molecule and a second group of atoms of a second element type in at least a part of the second molecule, an atomic pair composed of an atom in the first group of atoms and an atom in the second group of atoms; and determining an element value indexed by the first element type and the second element type in the predicted interaction matrix based on a weighted sum of representations of edges of atomic pairs.

In some embodiments, the method further includes: determining, for a first group of atoms of a first element type in the first molecule and a second group of atoms of a second element type in the second molecule, a number of one or more atomic pairs composed of an atom in the first group of atoms and an atom in the second group of atoms having a distance less than a third threshold; and determining an element value of a matrix element indexed by the first element type and the second element type in the real interaction matrix based on the number of the one or more atomic pairs.

In step 603, the prediction model is trained by minimizing a target loss function based on a difference between the predicted binding force and a real binding force and a difference between the predicted interaction matrix and a real interaction matrix.

In some embodiments, training the prediction model includes: determining a first loss function based on the difference between the binding force and the real binding force measured from an experiment; determining a second loss function based on the difference between the predicted interaction matrix and the real interaction matrix; and determining the target loss function based on a weighted sum of the first loss function and the second loss function.

In some embodiments, the first molecule is a ligand and the second molecule is a protein.

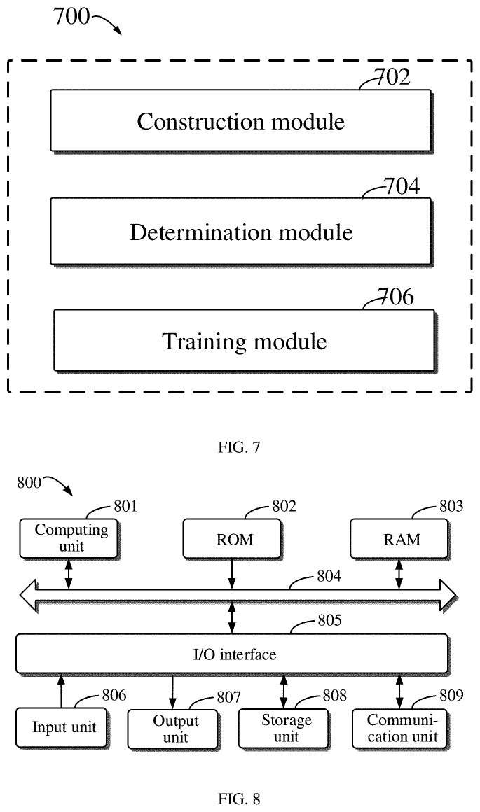

FIG. 7 shows a schematic block diagram of an apparatus 700 of training a prediction model according to an embodiment of the present disclosure. As shown in FIG. 7, the apparatus 700 includes a construction module 702 used to construct a virtual complex molecule based on a three-dimensional structure information of a first molecule and a second molecule. The virtual complex molecule includes a virtual representation of the first molecule and a virtual representation of at least a part of the second molecule. The apparatus 700 further includes a determination module 704 used to determine a predicted binding force between the first molecule and the second molecule and a predicted interaction matrix between the first molecule and the second molecule based on the virtual complex molecule by using the prediction model, the predicted interaction matrix indicates an element-type-based and distance-based interaction between an atom in the first molecule and an atom in the second molecule. The apparatus 700 further includes a training module 706 used to train the prediction model by minimizing a target loss function based on a difference between the predicted binding force and a real binding force and a difference between the predicted interaction matrix and a real interaction matrix. It should be understood that the construction module 702, the determination module 704, and the training module 706 may implement part or all of the functions of the pre-processing module 110, the graph neural network module 120, and the post-processing module 160 shown in FIG. 1.

In some embodiments, the construction module 702 includes: a distance determination sub-module used to determine a distance between a target atom in the second molecule and an atom in the first molecule based on the three-dimensional structure information; and an atom determination sub-module used to combine the target atom with the atom in the first molecule and determine the target atom and the atom in the first molecule as atoms of the virtual complex molecule, in response to determining that the distance between the target atom in the second molecule and the atom in the first molecule is less than a first threshold.

In some embodiments, the construction module 702 includes: an edge construction sub-module used to construct an edge between atoms having a distance less than a second threshold in the virtual complex molecule; and a representation determination sub-module used to determine a representation of an atom in the virtual complex molecule and a representation of the edge based on a three-dimensional structure information of the virtual complex molecule.

In some embodiments, the determination module 704 includes: a feature representation determination sub-module used to determine a feature representation for characterizing the virtual complex molecule based on the representation of the atom in the virtual complex molecule; and a predicted binding force determination sub-module configured to determine the predicted binding force based on the feature representation using a fully connected layer in the prediction mode.

In some embodiments, the determination module 704 includes: an atomic pair determination sub-module used to determine, for a first group of atoms of a first element type in the first molecule and a second group of atoms of a second element type in at least a part of the second molecule, an atomic pair composed of an atom in the first group of atoms and an atom in the second group of atoms; and a predicted interaction matrix determination sub-module used to determine an element value indexed by the first element type and the second element type in the predicted interaction matrix based on a weighted sum of representations of edges of atomic pairs.

In some embodiments, the apparatus 700 further includes: an atomic pair number determination module used to determine, for a first group of atoms of a first element type in the first molecule and a second group of atoms of a second element type in the second molecule, a number of one or more atomic pairs composed of an atom in the first group of atoms and an atom in the second group of atoms having a distance less than a third threshold; and a real interaction matrix determination module used to determine an element value of a matrix element indexed by the first element type and the second element type in the real interaction matrix based on the number of the one or more atomic pairs.

In some embodiments, the training module 706 includes: a first loss function determination module used to determine a first loss function based on the difference between the binding force and the real binding force measured from an experiment; a second loss function determination module used to determine a second loss function based on the difference between the predicted interaction matrix and the real interaction matrix; and a target loss function determination module used to determine the target loss function based on a weighted sum of the first loss function and the second loss function.

In some embodiments, the first molecule is a ligand and the second molecule is a protein.

In the technical solution of the present disclosure, the collection, storage, use, processing, transmission, provision, and disclosure of the user's personal information involved are in compliance with relevant laws and regulations, and do not violate public order and good customs.

According to an embodiment of the present disclosure, the present disclosure further provides an electronic device, a readable storage medium, and a computer program product.

FIG. 8 shows a block diagram of an exemplary electronic device 800 capable of implementing a method of training a prediction model for determining molecular binding force of an embodiment of the present disclosure. The electronic device is intended to represent various forms of digital computers, such as a laptop computer, a desktop computer, a workstation, a personal digital assistant, a server, a blade server, a mainframe computer, and other suitable computers. The electronic device may further represent various forms of mobile apparatuses, such as a personal digital assistant, a cellular phone, a smart phone, a wearable device, and other similar computing apparatuses. The components as illustrated herein, and connections, relationships, and functions thereof are merely examples, and are not intended to limit the implementation of the present disclosure described and/or required herein.

As shown in FIG. 8, the electronic device 800 includes a computing unit 801, which may perform various appropriate actions and processing based on a computer program stored in a read-only memory (ROM) 802 or a computer program loaded from a storage unit 808 into a random access memory (RAM) 803. Various programs and data required for the operation of the electronic device 800 may be stored in the RAM 803. The computing unit 801, the ROM 802 and the RAM 803 are connected to each other through a bus 804. An input/output (I/O) interface 805 is also connected to the bus 804.

Various components in the electronic device 800, including an input unit 806 such as a keyboard, a mouse, etc., an output unit 807 such as various types of displays, speakers, etc., a storage unit 808 such as a magnetic disk, an optical disk, etc., and a communication unit 809 such as a network card, a modem, a wireless communication transceiver, etc., are connected to the I/O interface 805. The communication unit 809 allows the electronic device 800 to exchange information/data with other devices through a computer network such as the Internet and/or various telecommunication networks.

The computing unit 801 may be various general-purpose and/or special-purpose processing components with processing and computing capabilities. Some examples of the computing unit 801 include but are not limited to a central processing unit (CPU), a graphics processing unit (GPU), various dedicated artificial intelligence (AI) computing chips, various computing units running machine learning model algorithms, a digital signal processor (DSP), and any appropriate processor, controller, microcontroller, and so on. The computing unit 801 may perform the various methods and processes described above, such as the method 400. For example, in some embodiments, the method 400 may be implemented as a computer software program that is tangibly contained on a machine-readable medium, such as the storage unit 808. In some embodiments, part or all of a computer program may be loaded and/or installed on the electronic device 800 via the ROM 802 and/or the communication unit 809. When the computer program is loaded into the RAM 803 and executed by the computing unit 801, one or more steps of the method 400 described above may be performed. Alternatively, in other embodiments, the computing unit 801 may be configured to perform the method 600 in any other appropriate way (for example, by means of firmware).

Various embodiments of the systems and technologies described herein may be implemented in a digital electronic circuit system, an integrated circuit system, a field programmable gate array (FPGA), an application specific integrated circuit (ASIC), an application specific standard product (ASSP), a system on chip (SOC), a complex programmable logic device (CPLD), a computer hardware, firmware, software, and/or combinations thereof. These various embodiments may be implemented by one or more computer programs executable and/or interpretable on a programmable system including at least one programmable processor. The programmable processor may be a dedicated or general-purpose programmable processor, which may receive data and instructions from the storage system, the at least one input apparatus and the at least one output apparatus, and may transmit the data and instructions to the storage system, the at least one input apparatus, and the at least one output apparatus.

Program codes for implementing the method of the present disclosure may be written in any combination of one or more programming languages. These program codes may be provided to a processor or a controller of a general-purpose computer, a special-purpose computer, or other programmable data processing apparatuses, so that when the program codes are executed by the processor or the controller, the functions/operations specified in the flowchart and/or block diagram may be implemented. The program codes may be executed completely on the machine, partly on the machine, partly on the machine and partly on the remote machine as an independent software package, or completely on the remote machine or server.

In the context of the present disclosure, the machine readable medium may be a tangible medium that may contain or store programs for use by or in combination with an instruction execution system, device or apparatus. The machine readable medium may be a machine-readable signal medium or a machine readable storage medium. The machine readable medium may include, but not be limited to, electronic, magnetic, optical, electromagnetic, infrared or semiconductor systems, devices or apparatuses, or any suitable combination of the above. More specific examples of the machine readable storage medium may include electrical connections based on one or more wires, portable computer disks, hard disks, random access memory (RAM), read-only memory (ROM), erasable programmable read-only memory (EPROM or flash memory), optical fiber, convenient compact disk read-only memory (CD-ROM), optical storage device, magnetic storage device, or any suitable combination of the above.

In order to provide interaction with the user, the systems and technologies described here may be implemented on a computer including a display apparatus (for example, a CRT (cathode ray tube) or LCD (liquid crystal display) monitor) for displaying information to the user, and a keyboard and a pointing apparatus (for example, a mouse or a trackball) through which the user may provide the input to the computer. Other types of apparatuses may also be used to provide interaction with users. For example, a feedback provided to the user may be any form of sensory feedback (for example, visual feedback, auditory feedback, or tactile feedback), and the input from the user may be received in any form (including acoustic input, voice input or tactile input).

The systems and technologies described herein may be implemented in a computing system including back-end components (for example, a data server), or a computing system including middleware components (for example, an application server), or a computing system including front-end components (for example, a user computer having a graphical user interface or web browser through which the user may interact with the implementation of the system and technology described herein), or a computing system including any combination of such back-end components, middleware components or front-end components. The components of the system may be connected to each other by digital data communication (for example, a communication network) in any form or through any medium. Examples of the communication network include a local area network (LAN), a wide area network (WAN), and the Internet.

The computer system may include a client and a server. The client and the server are generally far away from each other and usually interact through a communication network. The relationship between the client and the server is generated through computer programs running on the corresponding computers and having a client-server relationship with each other. The server may be a cloud server, a distributed system server, or a server combined with a blockchain.

It should be understood that steps of the processes illustrated above may be reordered, added or deleted in various manners. For example, the steps described in the present disclosure may be performed in parallel, sequentially, or in a different order, as long as a desired result of the technical solution of the present disclosure may be achieved. This is not limited in the present disclosure.

The above-described specific embodiments do not constitute a limitation on the scope of protection of the present disclosure. Those skilled in the art should understand that various modifications, combinations, sub-combinations and substitutions may be made according to design requirements and other factors. Any modifications, equivalent replacements and improvements made within the spirit and principles of the present disclosure shall be contained in the scope of protection of the present disclosure.

What is claimed is:

1. A method of training a prediction model for determining molecular binding force, comprising:
constructing a virtual complex molecule based on a three-dimensional structure information of a first molecule and a second molecule, wherein the virtual complex molecule comprises a virtual representation of the first molecule and a virtual representation of at least a part of the second molecule;
determining a predicted binding force between the first molecule and the second molecule and a predicted interaction matrix between the first molecule and the second molecule based on the virtual complex molecule by using the prediction model, wherein the predicted interaction matrix indicates an element-type-based and distance-based interaction between an atom in the first molecule and an atom in the second molecule; and
training the prediction model by minimizing a target loss function based on a difference between the predicted binding force and a real binding force and a difference between the predicted interaction matrix and a real interaction matrix.

2. The method according to claim 1, wherein the constructing a virtual complex molecule comprises:
determining a distance between a target atom in the second molecule and an atom in the first molecule based on the three-dimensional structure information; and
combining the target atom with the atom in the first molecule and determining the target atom and the atom in the first molecule as atoms of the virtual complex molecule, in response to determining that the distance between the target atom in the second molecule and the atom in the first molecule is less than a first threshold.

3. The method according to claim 1, wherein the constructing a virtual complex molecule comprises:
constructing an edge between atoms having a distance less than a second threshold in the virtual complex molecule; and
determining a representation of an atom in the virtual complex molecule and a representation of the edge based on a three-dimensional structure information of the virtual complex molecule.

4. The method according to claim 3, wherein the determining a predicted binding force between the first molecule and the second molecule comprises:
determining a feature representation for characterizing the virtual complex molecule based on the representation of the atom in the virtual complex molecule; and
determining the predicted binding force based on the feature representation using a fully connected layer in the prediction model.

5. The method according to claim 3, wherein the determining a predicted interaction matrix comprises:
determining, for a first group of atoms of a first element type in the first molecule and a second group of atoms of a second element type in at least a part of the second molecule, an atomic pair composed of an atom in the first group of atoms and an atom in the second group of atoms; and
determining an element value indexed by the first element type and the second element type in the predicted interaction matrix based on a weighted sum of representations of edges of atomic pairs.

6. The method according to claim 1, further comprising:
determining, for a first group of atoms of a first element type in the first molecule and a second group of atoms of a second element type in the second molecule, a number of one or more atomic pairs composed of an atom in the first group of atoms and an atom in the second group of atoms having a distance less than a third threshold; and
determining an element value of a matrix element indexed by the first element type and the second element type in the real interaction matrix based on the number of the one or more atomic pairs.

7. The method according to claim 1, wherein the training the prediction model comprises:
determining a first loss function based on the difference between the binding force and the real binding force measured from an experiment;
determining a second loss function based on the difference between the predicted interaction matrix and the real interaction matrix; and
determining the target loss function based on a weighted sum of the first loss function and the second loss function.

8. The method according to claim 1, wherein the first molecule is a ligand and the second molecule is a protein.

9. The method according to claim 2, wherein the constructing a virtual complex molecule comprises:
constructing an edge between atoms having a distance less than a second threshold in the virtual complex molecule; and
determining a representation of an atom in the virtual complex molecule and a representation of the edge based on a three-dimensional structure information of the virtual complex molecule.

10. The method according to claim 2, further comprising:
determining, for a first group of atoms of a first element type in the first molecule and a second group of atoms of a second element type in the second molecule, a number of one or more atomic pairs composed of an atom in the first group of atoms and an atom in the second group of atoms having a distance less than a third threshold; and
determining an element value of a matrix element indexed by the first element type and the second element type in the real interaction matrix based on the number of the one or more atomic pairs.

11. An electronic device, comprising:
at least one processor; and
a memory communicatively connected to the at least one processor,
wherein the memory stores instructions executable by the at least one processor, and the instructions, when executed by the at least one processor, cause the at least one processor to implement operations of training a prediction model for determining molecular binding force, comprising:
constructing a virtual complex molecule based on a three-dimensional structure information of a first molecule and a second molecule, wherein the virtual complex molecule comprises a virtual representation of the first molecule and a virtual representation of at least a part of the second molecule;
determining a predicted binding force between the first molecule and the second molecule and a predicted interaction matrix between the first molecule and the second molecule based on the virtual complex molecule by using the prediction model, wherein the predicted interaction matrix indicates an element-type-based and distance-based interaction between an atom in the first molecule and an atom in the second molecule; and
training the prediction model by minimizing a target loss function based on a difference between the predicted binding force and a real binding force and a difference between the predicted interaction matrix and a real interaction matrix.

12. The electronic device according to claim 11, wherein the instructions, when executed by the at least one processor, further cause the at least one processor to:
determine a distance between a target atom in the second molecule and an atom in the first molecule based on the three-dimensional structure information; and
combine the target atom with the atom in the first molecule and determine the target atom and the atom in the first molecule as atoms of the virtual complex molecule, in response to determining that the distance between the target atom in the second molecule and the atom in the first molecule is less than a first threshold.

13. The electronic device according to claim 11, wherein the instructions, when executed by the at least one processor, further cause the at least one processor to:
construct an edge between atoms having a distance less than a second threshold in the virtual complex molecule; and
determine a representation of an atom in the virtual complex molecule and a representation of the edge based on a three-dimensional structure information of the virtual complex molecule.

14. The electronic device according to claim 13, wherein the instructions, when executed by the at least one processor, further cause the at least one processor to:
determine a feature representation for characterizing the virtual complex molecule based on the representation of the atom in the virtual complex molecule; and
determine the predicted binding force based on the feature representation using a fully connected layer in the prediction model.

15. The electronic device according to claim 13, wherein the instructions, when executed by the at least one processor, further cause the at least one processor to:
determine, for a first group of atoms of a first element type in the first molecule and a second group of atoms of a second element type in at least a part of the second molecule, an atomic pair composed of an atom in the first group of atoms and an atom in the second group of atoms; and
determine an element value indexed by the first element type and the second element type in the predicted interaction matrix based on a weighted sum of representations of edges of atomic pairs.

16. The electronic device according to claim 11, wherein the instructions, when executed by the at least one processor, further cause the at least one processor to:
determine, for a first group of atoms of a first element type in the first molecule and a second group of atoms of a second element type in the second molecule, a number of one or more atomic pairs composed of an atom in the first group of atoms and an atom in the second group of atoms having a distance less than a third threshold; and
determine an element value of a matrix element indexed by the first element type and the second element type in the real interaction matrix based on the number of the one or more atomic pairs.

17. The electronic device according to claim 11, wherein the instructions, when executed by the at least one processor, further cause the at least one processor to:
determine a first loss function based on the difference between the binding force and the real binding force measured from an experiment;
determine a second loss function based on the difference between the predicted interaction matrix and the real interaction matrix; and
determine the target loss function based on a weighted sum of the first loss function and the second loss function.

18. The electronic device according to claim 11, wherein the first molecule is a ligand and the second molecule is a protein.

19. The electronic device according to claim 12, wherein the instructions, when executed by the at least one processor, further cause the at least one processor to:
construct an edge between atoms having a distance less than a second threshold in the virtual complex molecule; and
determine a representation of an atom in the virtual complex molecule and a representation of the edge based on a three-dimensional structure information of the virtual complex molecule.

20. A non-transitory computer-readable storage medium having computer instructions therein, wherein the computer instructions are configured to cause a computer to implement operations of training a prediction model for determining molecular binding force, comprising:
constructing a virtual complex molecule based on a three-dimensional structure information of a first molecule and a second molecule, wherein the virtual complex molecule comprises a virtual representation of the first molecule and a virtual representation of at least a part of the second molecule;
determining a predicted binding force between the first molecule and the second molecule and a predicted interaction matrix between the first molecule and the second molecule based on the virtual complex molecule by using the prediction model, wherein the predicted interaction matrix indicates an element-type-based and distance-based interaction between an atom in the first molecule and an atom in the second molecule; and
training the prediction model by minimizing a target loss function based on a difference between the predicted binding force and a real binding force and a difference between the predicted interaction matrix and a real interaction matrix.
